(19) United States
(12) Patent Application Publication
(10) Pub. No.: US 2019/0120827 A1
(43) Pub. Date: Apr. 25, 2019

(54) METHODS AND COMPOSITIONS FOR NICOTINIC RECEPTOR ASSAYS

(71) Applicant: GEORGE MASON UNIVERSITY, Fairfax, VA (US)

(72) Inventor: Nadine Kabbani, Washington, DC (US)

(21) Appl. No.: 16/142,494

(22) Filed: Sep. 26, 2018

Related U.S. Application Data
(60) Provisional application No. 62/563,150, filed on Sep. 26, 2017.

(51) Int. Cl. G01N 33/50; G01N 33/58

(52) U.S. Cl. CPC G01N 33/5041; G01N 2333/70571; G01N 33/582

(57) ABSTRACT

Methods and compositions for assays of the cellular function of G-protein coupled nicotinic receptors, for example, the alpha 7 nicotinic acetylcholine receptor, a7nAChR. G-protein coupled functions of this receptor are found in various types of cells including, but not limited to, neural, immune, and cancer cells. The assays disclosed herein find a basis in the specificity of the a7nAChR/G protein signaling and the ability to directly survey this activity using fluorescent probes and genetic molecular tools.

### CROSS REFERENCE TO RELATED APPLICATIONS

This application claims priority to, the benefit under 35 U.S.C. § 119 of, and incorporates by reference herein in its entirety U.S. Provisional Patent Application No. 62/563,150, filed Sep. 26, 2017, and entitled “Methods and Compositions for Nicotinic Receptor Assays.”

### FIELD OF THE DISCLOSURE

The present disclosure relates to receptor/reporter fusion protein based assays for detecting an effect that test compounds have on a particular membrane receptor, as well as to receptor/reporter fusion proteins for use in such assays and compounds identified by the assays as having interesting/useful effects.

### BACKGROUND

Traditional protocols for the measurement of ligand activity at receptors such as G-protein coupled receptors (GPCRs) have relied upon a number of biochemical techniques. These include radioligand binding analysis in which the ability of a test compound to displace the binding of a known radioligand is determined, and a number of functional assays in which the ability of a test compound to activate or inhibit a specific signal transduction event is measured.

Functional assays of ligand activity at GPCRs expressed in mammalian cells include the measurement of the rate of guanine nucleotide exchange at the activated G-protein alpha sub-unit, the measurement of the changes in the level of one of an assortment of intracellular second messenger metabolites, such as cAMP, calcium, or inositol phosphates, or the activation or inhibition of an ion channel.

Nicotinic acetylcholine receptors (nAChRs) comprise an important class of the Cys-loop ligand-gated ion channel super-family, which mediate communication between neurons by conversion of chemical neurotransmitter signals into a trans-membrane flux of ions. In most cell types, co-expression of ionotropic nAChRs as well as metabotropic muscarinic receptors ensures a fast and slow acetylcholine signaling response, respectively. At least nine different nAChR subunits are expressed in the mammalian brain. In the hippocampus and cortex homomeric α7 and heteromeric α4β2 nAChRs have been shown to contribute to neurotransmitter release and dendritic plasticity. Upon ligand activation, α7 nAChRs conduct cations into the cell.

What is needed are methods and compositions for assays for detecting compounds that affect the G-protein signaling activities of nAChRs.

### BRIEF SUMMARY

Disclosed are methods of assaying the effect of compounds on the G-protein signaling activities of nAChRs. Further disclosed are compositions comprising components of assays for detecting and measuring the effect of assayed compounds on the G-protein signaling activities of nAChRs.

In an aspect the disclosure provides an assay for detecting an effect a test compound or molecule has on a membrane receptor/coupled protein, comprising the steps of: a) adding the test compound to a cell comprising a disclosed, optionally one or more proteins are labeled, membrane receptor/coupled protein; and b) detecting any change of said receptor/coupled protein.

In an aspect the disclosure provides an assay for detecting an effect a test compound has on a membrane receptor/coupled protein, comprising the steps of: a) adding the compound to a cell comprising a disclosed membrane receptor protein coupled to at least one G protein; and b) detecting any change of the receptor and/or the at least one G protein.

Disclosed assays may be used to screen compounds for their effect on particular membrane receptors. Compounds identified as having an effect on a particular membrane receptor may be useful, for example, in modulating the activity of wild type and/or mutant membrane receptors; may be used in elaborating the biological function of particular membrane receptors; and/or may be used in screens for identifying compounds that disrupt normal membrane receptor interactions, or can in themselves disrupt such interactions.

Disclosed assays may be particularly suited for the detection of compounds which serve as inverse agonists, antagonists, agonists, or allosteric modulators of the membrane receptor. The term inverse agonist is understood to mean a compound which when it binds to a receptor, selectively stabilizes and thus enriches the proportion of a receptor in a conformation or conformations that are incapable of inducing a downstream signal. Agonist is understood to mean a compound which when it binds to a receptor selectively stabilizes and thus enriches the proportion of the receptor in a conformation or conformations capable of inducing a downstream signal. Antagonist is understood to mean a compound which when it binds to a receptor has no selective ability to enrich either active or inactive conformations and thus does not alter the equilibrium between them. Allosteric modulator is a compound or substance, which indirectly influences (modulates) the effects of an agonist or inverse agonist at the receptor site.

In one embodiment, an assay for detecting an effect a test agent has on a membrane receptor, comprising the steps of: a) adding a test agent to a cell expressing a G-protein coupled membrane receptor/reporter protein coupled to one or more G proteins, wherein the receptor/reporter protein comprises a membrane receptor segment and a reporter segment comprising a first pair of reporter molecules, and wherein at least one of the one or more G proteins comprises a second pair of reporter molecules; and b) detecting the signal from the first and/or second pair of reporter molecules, wherein a change in the signal indicates a change of coupling of the receptor/reporter protein and the at least one of the one or more G proteins.

In one embodiment, an assay for detecting a test agent which has an effect on a membrane receptor, comprising the steps of: a) expressing a G-protein coupled membrane receptor/reporter protein capable of coupling to one or more G proteins in a cell, wherein the receptor/reporter protein comprises a membrane receptor segment and a reporter segment and wherein each of the one or more G proteins are labeled with a reporter molecule; b) detecting a basal activity level of the reporter molecules; c) adding a test agent to the cell; and d) detecting a resulting activity level of the reporter molecules; and e) comparing the basal activity level with the resulting activity level to determine whether alteration of the basal activity level has occurred, wherein the alteration is due to the test agent having an effect on the membrane receptor segment and/or the coupling with G proteins.

According to one aspect of the present disclosure, the assay, wherein the membrane receptor segment is the alpha7 nicotinic receptor and its variants. Furthermore, the assay, wherein the first pair of reporter molecules is YFP or CFP. Moreover, the assay, wherein the second pair of reporter molecules is CFP or YFP. Further, the assay, wherein the assay is further used to screen agents for their effect on membrane receptors. Additionally, the assay, wherein the assay is further used to identify agents that disrupt normal membrane receptor interactions. Also, the assay, wherein the test agent serves as an inverse agonist, antagonist, agonist, or allosteric modulator of the membrane receptor.

According to one aspect of the present disclosure, the assay, wherein the inverse agonist, antagonist, agonist, or allosteric modulator of the membrane receptor is used in the study of receptor function. Furthermore, the assay, wherein the receptor/reporter protein is expressed from nucleic acid construct comprising a gene encoding the reporter segment that is fused in-frame to the 5′ or 3′ end of a gene encoding the membrane receptor segment. Moreover, the assay, wherein the functionality of the membrane receptor segment is substantially unaffected by the presence of the reporter segment or a reporter molecule on the membrane receptor segment. Further, the assay, wherein the signal is detected by FRET or BRET.

According to one aspect of the present disclosure, the assay, wherein the membrane receptor segment is the alpha7 nicotinic receptor and its variants. Additionally, the assay, wherein the reporter molecule is CFP or YFP. Also, the assay, wherein the test agent serves as an inverse agonist, antagonist, agonist, or allosteric modulator of the membrane receptor. Furthermore, the assay, wherein the inverse agonist, antagonist, agonist, or allosteric modulator of the membrane receptor is used in the study of receptor function. Moreover, the assay, wherein the receptor/reporter protein is expressed from nucleic acid construct comprising a gene encoding the reporter segment that is fused in-frame to the 5′ or 3′ end of a gene encoding the membrane receptor segment. Further, the assay, wherein the functionality of the membrane receptor segment is substantially unaffected by the presence of the reporter segment or the reporter molecules. Additionally, the assay, wherein the detecting of steps b) and d) is detected by BRET. Also, the assay, wherein the detecting of steps b) and d) is detected by FRET.

According to additional aspects of the present disclosure, the assay, wherein each of the one or more G proteins comprises the second pair of reporter molecules. Furthermore, the assay, wherein the signal is detected from both the first and second pair of reporter molecules. Moreover, the assay, wherein the change in signal indicates a change of coupling of the receptor/reporter proteins and each of the one or more G proteins.

The term compound is understood to include chemicals (such as small molecules) as well as peptides and/or proteins. Compounds may be introduced exogenously or endogenously (through genetic delivery). In some aspects, peptides maybe introduced through genetic transfection where the cell will make them.

The present disclosure also therefore relates to inverse agonists, antagonists, agonists, or allosteric modulators of receptor proteins identified using the assays according to the present disclosure and to the use of such agonists, antagonists, agonists, or allosteric modulators in studying receptor function, or therapy.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the disclosure as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain and illustrate the principles of the disclosed method and compositions. Although not specifically referenced below in the Detailed Description, one having ordinary skill in the art would understand and appreciate the applicability of the accompanying drawings to the disclosed receptor/reporter fusion protein based assays for detecting an effect that test compounds have on a particular membrane receptor, as well as to the disclosed receptor/reporter fusion proteins for use in such assays and compounds identified by the assays as having interesting/useful effects. Regardless, to assist with understanding the applicability of the accompanying drawings to the Detailed Description, the following brief descriptions may be useful:

FIG. 1 (consisting of FIGS. 1A and 1B) shows protein binding motifs for G protein binding across species, according to one embodiment of the present disclosure. Protein-protein binding motifs responsible for G protein binding were discovered and characterized within the alpha 7 nicotinic receptor. Based on sequence identify at this site, the G protein binding motifs are conserved across various organisms (FIG. 1A) and shared with a subset of nicotinic receptors (FIG. 1B);

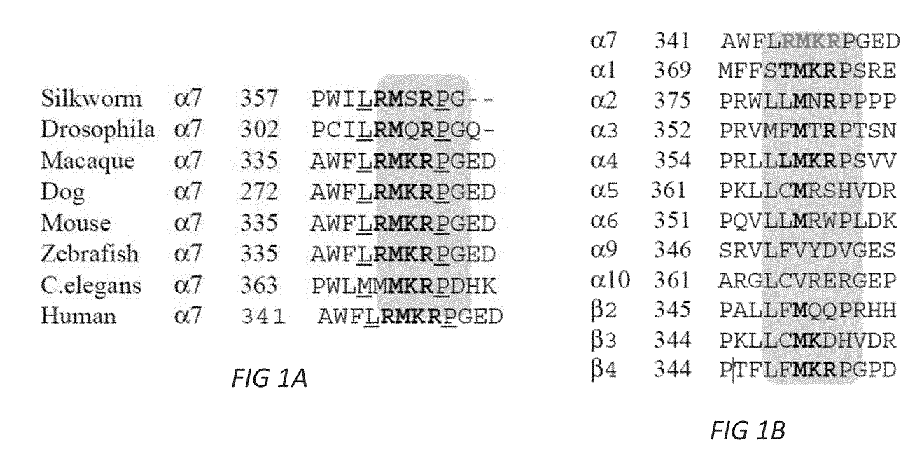

FIG. 2 shows protein binding motifs for G protein binding across a subset of nicotinic receptors, according to one embodiment of the present disclosure. Generally, amino Acids within the nicotinic receptor involved in G protein interaction are shown boxed;

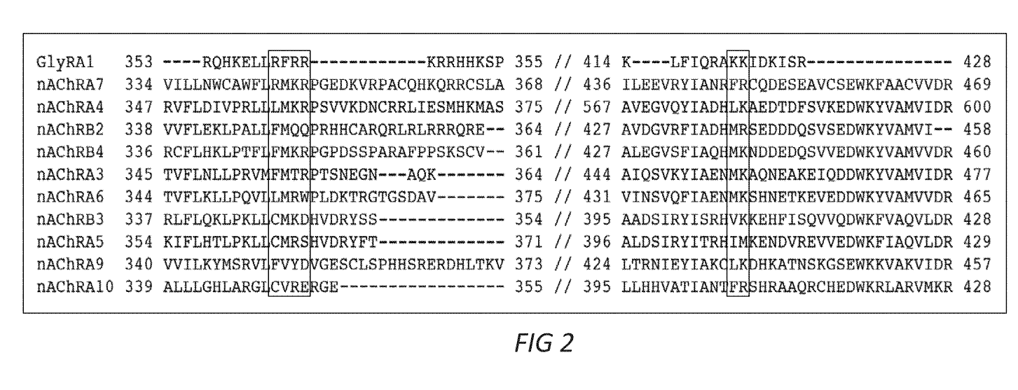

FIG. 3 shows ligand activation of the alpha 7 nAChR in is associated with G protein, according to one embodiment of the present disclosure. Generally, ligand activation of the alpha 7 nAChR in various types of cells is associated with a novel mode of signaling dependent on G protein activity, which can be exploited for the screening and identification of new compounds that can target the ability of the alpha 7 nAChR to activate G proteins;

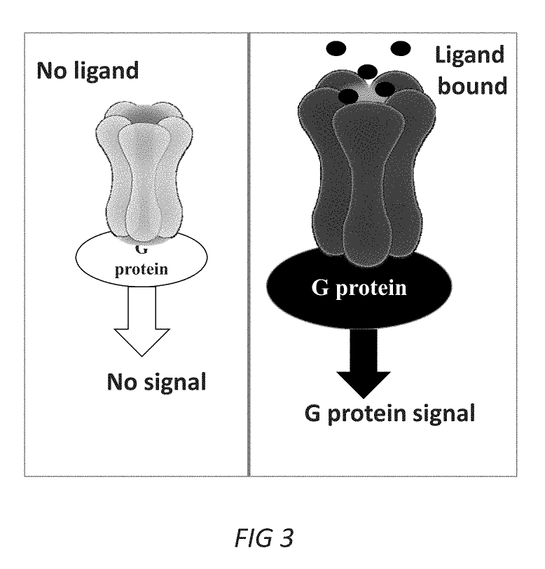

FIG. 4 shows an exemplary assay, according to one embodiment of the present disclosure;

FIG. 5 (consisting of FIGS. 5A and 5B) shows an exemplary series of FRET pairs for the detection of alpha 7 nicotinic receptor, according to one embodiment of the present disclosure;

FIG. 6 (consisting of FIGS. 6A-6C) shows nucleic acid constructs, sequences for FRET pairs for the detection of alpha 7 nicotinic receptor activation of G protein signaling, and a YFP sequence (noted with “*” in FIG. 6C) inserted within the M3-M4 loop region of the human alpha 7 nicotinic receptor, according to one embodiment of the present disclosure;

FIG. 7 (consisting of FIGS. 7A-7C) shows nucleic acid constructs, sequences for FRET pairs for the detection of alpha 7 nicotinic receptor activation of G protein signaling, and a CFP sequence (noted with “*” in FIG. 7C) inserted within the M3-M4 loop region of the human alpha 7 nicotinic receptor, according to one embodiment of the present disclosure;

FIG. 8 (consisting of FIGS. 8A and 8B) shows BRET pairs for the detection of alpha 7 nicotinic receptor activation of G protein signaling, according to one embodiment of the present disclosure;

FIG. 9 (consisting of FIGS. 9A-9C) shows nucleic acid constructs, sequences for BRET pairs for the detection of alpha 7 nicotinic receptor activation of G protein signaling, and an EYFP sequence (noted with “*” in FIG. 9C) inserted within the M3-M4 loop region of the human alpha 7 nicotinic receptor, according to one embodiment of the present disclosure;

FIG. 10 (consisting of FIGS. 10A-10C) nucleic acid constructs, sequences for BRET pairs for the detection of alpha 7 nicotinic receptor activation of G protein signaling, and a Rluc sequence (noted with “*” in FIG. 10C) inserted within the M3-M4 loop region of the human alpha 7 nicotinic receptor, according to one embodiment of the present disclosure; and

FIG. 11 (consisting of FIGS. 11A-11C and FIGS. 11A′-11C′) shows a schematic of the detection of G protein activation. BRET/FRET compatible heterotrimeric G proteins (α, β, γ). FIGS. 11A-11C illustrate a series of possible BRET/FRET sensors that can be used in measures of G protein activation by the alpha 7 nicotinic receptor (nAChR), according to one embodiment of the present disclosure.

### DETAILED DESCRIPTION

The disclosed methods and compositions may be understood more readily by reference to the following detailed description of particular embodiments and the Example included therein and to the Figures and their previous and following descriptions.

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present disclosure which will be limited only by the appended claims.

Disclosed herein are methods and compositions for assays of the cellular function of G-protein coupled nicotinic receptors, for example, the alpha 7 nicotinic acetylcholine receptor, a7nAChR. G-protein coupled functions of this receptor is found in various types of cells including, but not limited to, neural, immune, and cancer cells. Assays disclosed herein find a basis in the specificity of the a7nAChR/G protein signaling and the ability to directly survey this activity using fluorescent probes and genetic molecular tools.

Disclosed are compositions comprising receptors and proteins coupled with a receptor, which includes compositions comprising amino acid sequences (e.g., peptides and proteins) and compositions comprising nucleic acids encoding and/or expressing such amino acid sequences. Disclosed compositions comprise nucleic acid constructs, vectors comprising disclosed nucleic acids, cells comprising disclosed nucleic acids, or disclosed vectors, and cell comprising disclosed peptides and proteins.

The present disclosure relates to G protein coupled a7nACh receptor, and the DNA sequences encoding such receptors, and these and other disclosed components may be used in assays of the present disclosure. Other nicotinic ACh receptors are contemplated in the disclosure, and the disclosure is not to be seen as limited by reference to G protein coupled a7nACh receptor for the sake of brevity.

GPCRs can exist as monomers, dimers, or heterodimers, when expressed in mammalian cells. The ability of GPCRs to form heterodimers provides a novel mechanism of cellular signaling. Two GPCRs that heterodimerize or one GPCR and a receptor that binds to the GPCR can attain signaling functions and ligand binding functions that are distinct from when only one of the receptors is present in a cell. As indicated above, the GPCRs are important to the functioning of a cell. Where the GPCR activation results in the regulation of another GPCR expressed on the same cell, there is interest in being able to detect and modulate the dimer- or oligomerization. By inhibiting the complexing of the GPCR with another membrane protein necessary for signal transduction, one can affect the pathway(s) regulated by the GPCR and the pathway(s) affected by the second membrane protein. There is substantial interest in determining the effect of ligand binding to a GPCR, as well as the formation of a heterodimeric GPCR, complex on cell pathways.

In view of the importance of the GPCRs on the physiological status of mammals, there has been substantial interest in developing compounds that can modulate the activity of specific GPCRs and the interaction of GPCRs with other proteins in the cellular membrane and in the cytosol.

The most commonly used system of classification of GPCRs is that implemented in the GPCRDB database which may be found on the world wide web at gper-dot-org. It divides the GPCRs into six classes (Class A: Rhodopsin-like, with over 80% of all GPCRs in humans; Class B: Secretin-like; Class C: Metabotropic glutamate receptors; Class D: Pheromone receptors; Class E: cAMP receptors; and the much smallerClassFirzzled/smoothened family) Classes A, B, C and F are found in mammalian species while Class D proteins are found only in fungi and Class E proteins are exclusive to Dictyostelium. The six classes are further divided into sub-divisions and sub-sub-divisions based on the function of a GPCR and its specific ligand Nicotinic receptors, such as alpha7nACHRs, represent a novel class of G protein acting receptors, which operate with similarity to GPCRs.G Proteins.

G protein-coupled receptors (GPCRs) form one of the largest families of integral membrane receptors. GPCRs transduce information provided by extracellular stimuli into intracellular second messengers via their coupling to heterotrimeric G proteins and the subsequent regulation of a variety of effector systems. Common to most GPCRs is the cyclic process of signaling, desensitization, internalization, resensitization, and recycling to the plasma membrane. This cycle prevents cells from undergoing excessive receptor stimulation or periods of prolonged inactivity.

Disclosed herein are compositions comprising receptors and associated or coupled proteins, such as G proteins. Compositions may comprise labeled receptors, and/or labeled associated or coupled proteins, such as G proteins. As used herein, coupled or associated are used interchangeably to refer to proteins, such as G proteins, that interact with or are affected by or acted on, e.g., are coupled with receptors, such as nAChRs, disclosed herein. In a composition or method disclosed herein, a G protein may be coupled or associated with a receptor or receptor subunit that it is normally found with, or a G protein may be coupled or associated with a receptor it is not normally associated with, e.g., not found commonly within a particular cell.

Membrane receptor channels or coupled G proteins disclosed herein may be modified by the fusion (linked at the nucleic acid level) or by covalently bonding of a reporter protein to a receptor and/or to one or more coupled proteins, such as G proteins. For example, a nucleic acid encoding the reporter protein and/or coupled proteins, such as G protein may be fused in-frame to an end, that is the 5′ or 3′ end, of a gene encoding the particular receptor and/or. In this manner, on expression of the gene, the reporter protein and/or G protein is functionally expressed and fused to the N-terminal or C-terminal end of the receptor and/or G protein. Modification of a receptor and/or coupled proteins, such as G protein, is such that the functionality of the membrane receptor and/or coupled proteins, such as G protein remains substantially unaffected by fusion of the reporter protein to the receptor and/or G protein. Alternatively, a reporter molecule may be covalently linked to the receptor and/or coupled proteins, such as G protein. Modification of the receptor and/or coupled proteins, such as G protein by covalent linkage of the reporter does not impede the functionality and the membrane receptor and/or coupled proteins, such as G protein remains substantially unaffected by fusion of a reporter protein to the receptor and/or coupled proteins, such as G protein.

The nucleic acid constructs of the present disclosure comprise nucleic acid, typically DNA, encoding the particular label (detectable molecule) to which is fused, in-frame, the appropriate gene encoding the receptor or coupled protein. Generally speaking the nucleic acid constructs are expressed in the cells by means of an expression vector. Typically, although not exclusively the cells are of mammalian origin and the expression vector chosen is one which is suitable for expression in the particular cell type.

An expression vector is a replicable DNA construct in which the nucleic acid is operably linked to suitable control sequences capable of effecting the expression of the membrane receptor/reporter fusion in the particular cell. Typically control sequences may include a transcriptional promoter, an optional operator sequence to control transcription, a sequence encoding suitable mRNA ribosomal binding sites, and sequences which control the termination of transcription and/or translation. Typical expression vectors may include for example plasmids, bacteriophages or viruses and such vectors may integrate into the host's genome or replicate automonously in the particular cell.

In order for the particular cell to express a receptor/reporter fusion protein or a coupled protein/reporter fusion protein, the cell must be transformed by the appropriate expression vector. “Transformation”, as used herein, refers to the introduction of a heterologous polynucleotide fragment into a host cell, irrespective of the method used, for example direct uptake, transfection or transduction.

The present disclosure comprise cells which have been transformed by nucleic acid constructs comprising receptors and/or G proteins disclosed herein and/or receptor/reporter and/or G proteins fusions disclosed and express the receptor/reporter and/or G proteins fusion protein.

An aspect of the present disclosure relates to host cells comprising vectors containing nucleic acid sequences, and expression vectors. Vectors disclosed herein and known to those of skill in the art are provided, where the nucleotide sequence is operatively linked to and under the control of regulatory nucleotide sequences which are likewise present in the vector and which are arranged within the nucleotide sequence. These regulatory nucleotide sequences may be heterologous to the nucleotide sequence of receptors and/or coupled proteins, i.e., they may be derived from a different organism or from a different gene, or homologous, i.e., naturally occurring together with the nucleotide sequences of the disclosure in a regulatory unit.

The recombinant expression vectors of the disclosure comprise a nucleic acid in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like.

The recombinant expression vectors can be designed for expression of the proteins in prokaryotic or eukaryotic cells. For example, the proteins can be expressed in bacterial cells such as Escherichia coli, insect cells (using baculovirus expression vectors) yeast cells or mammalian cells.

The present disclosure comprises cells which express receptors and/or G proteins disclosed herein and/or labeled receptor/reporter and/or G proteins disclosed.

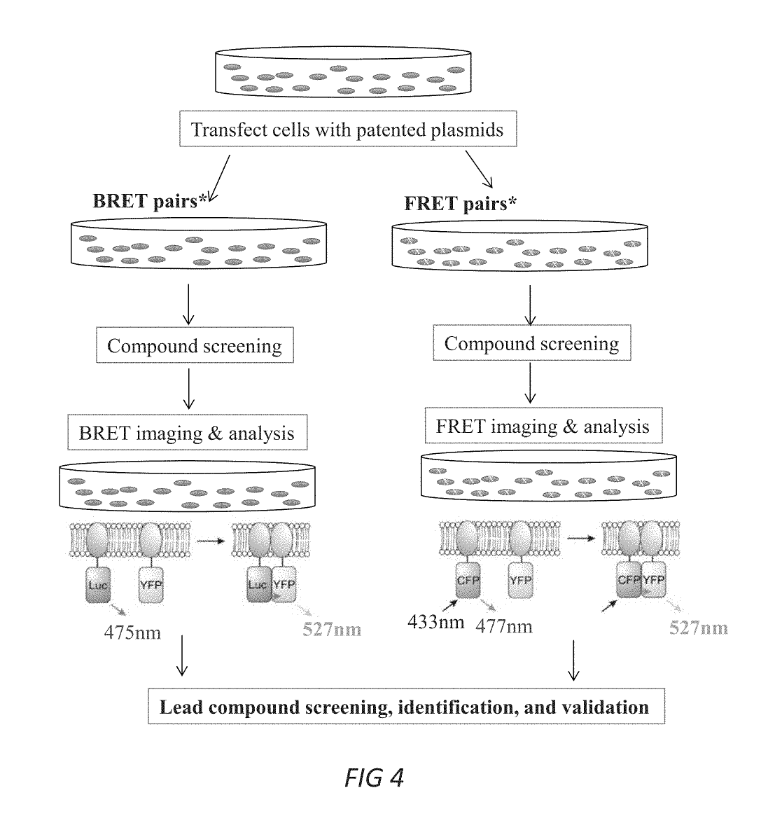

### Methods (See, e.g., FIG. 4).

In certain aspects, disclosed herein is a method for determining activation of a cell surface receptor, said cell surface receptor being one which binds to an associated or coupled protein, which may be an intracellular protein. A method comprises the step of providing a cell expressing or comprising at least one of (i) a cell surface receptor or receptor subunit, for example, alpha 7nAChR; (ii) one or more labelled intracellular proteins, such as a G protein. A method comprises a step of contacting the receptor on the cell with an agent that can be, for example, an agonist, antagonist, or allosteric modulator. This may be referred to as a ligand, and the contact is for a sufficient time for the ligand to bind to the receptor and for any binding or release of one or more intracellular associated or coupled proteins to the receptor to occur. The method then comprises steps, which measure any change to the receptor, one or more associated or coupled proteins such as the G protein, or both.

In certain aspects, a method comprises determining activation by a ligand of the alpha 7nAChR. A method may comprise the steps of (a) providing cells expressing (i) a labeled or not labeled alpha 7nAChR, (ii) one or more labeled or not labeled G protein; (b) contacting the cells with a ligand for sufficient time for the ligand to bind to the alpha 7nAChR; (c) determining a change in one or more labels, wherein a change indicates activation of the alpha 7nAChR.

In certain aspects of the disclosure there is proved a kit for use in receptor such as the alpha 7nAChR assays. The kit may comprise a genetic construct, such as an expression vector, for transforming cells, said construct encoding one or more G proteins. The kit may further comprise a genetic construct encoding a receptor such as the alpha 7nAChR. A kit may comprise cells comprising labeled or unlabeled G proteins wherein the G proteins are labeled by covalently attached labels, and/or labeled or unlabeled receptors such as the alpha 7nAChR.

Cellular membrane proteins fulfill many functions in transducing signals when ligand binds, acting as ion channels, binding to other proteins involving diapedesis, viral nucleic acid insertion, immune synapse, etc. For many receptors of clinical interest, upon binding to ligand, the cellular membrane receptor becomes endocytosed, so that the population at the surface may change in the presence of ligand or an agonist. These proteins are typically endocytosed to a greater degree after activation.

Many cell surface receptors upon binding to a ligand may then bind to an auxiliary protein and may endocytose and may become associated with an endosome. Such cell surface receptors often bind G proteins and are an important class of receptors of interest for pharmaceutical therapies.

Membrane receptors are activated by an external (and sometimes an internal) signal resulting in a conformational change. once the receptor becomes bound it activates the G protein leading to a change in its signaling. Many G proteins are heterotrimers, which upon activation convert GDP to GTP. The G protein signal may persist for various durations and is often turned off by other cell factors such as phosphorylation of the receptor by kinases.

Methods and Compositions for Assays

In performing disclosed methods, cells are incubated in an assay medium in a selected environment, normally including an agonist or agonist candidate, for sufficient time for any binding to occur, and, optionally, for endocytosis to reach equilibrium (typically about 90 min.), followed by detecting a signal, and then determining the signal as a measure of the binding. In some instances, one may study an antagonist for displacing or preventing binding of the agonist or a receptor-modulating compound.

Disclosed assays are performed under standard cell culture conditions. Depending upon the mode of the assay, different selected environmental conditions may be employed. For studying ligands, the selected environment will include a candidate ligand to detect any resulting activity.

Cells Used in Assay

Any eukaryotic cell may be employed, for the most part cell lines being employed. The cell lines will usually be mammalian, but for some purposes unicellular organisms or cells from non-vertebrates can be used. Mammalian cell lines include CHO, HeLa, MMTV, HepG2, HEK, U2OS, PC12 and the like. The cells may be genetically modified transiently or permanently. Various vectors that are commercially available can be used successfully to introduce the expression constructs into the cell. For an extensive description of cell lines, vectors, methods of genetic modification, and expression constructs, see published US application serial no. 2003/0092070, Zhao, et al., May 15, 2003, paragraphs 00046-00066, which are also specifically incorporated herein by reference.

Transformed (aka transfected) cells may be cloned that have various expression levels of the proteins. For example, the best clone may be chosen by lowest EC50 and best signal to background ratio. The cells are transiently or permanently transformed, in the case of the former using a conventional vector, normally a viral vector, e.g., adenovirus or Moloney Murine Leukemia Virus. Methods include transfection, electroporation, microinjection, transduction, cell fusion, DEAE dextran, calcium phosphate precipitation, lipofection (lysosome fusion), use of a gene gun, using a viral vector, with a DNA vector transporter, and the like. For permanent insertion into the genome, various techniques are available for the insertion of the sequence in a homologous or non-homologous fashion. These techniques are well known. For random insertion, the introduction of the nucleic acid by any of the above methods will usually be sufficient. For homologous recombination, see, for example, U.S. Pat. Nos. 7,361,641, 5,578,461, 5,272,071 and PCT/US92/09627.

A screening assay method involves growing the cells in an appropriate medium and then adding the test agent. Typically, the medium is then incubated for at least about 0.25 h and not more than about 12 h. The volume will generally not exceed about 250 μl, usually not more than about 200 μl, and generally be at least about 10 μl, more usually at least about 20 μl, where the volume of the test agent solution addition will generally dilute the cell medium less than about 1:1, usually not more than about 0.5:1. When the reagent is dry, there will be no dilution.

The detection of ligand binding to proteins is important in many different areas of biology and medicine. Particularly, during the development of chemical compounds into drugs, it is important to know if the compound interacts with the drug target. The monitoring of target protein-ligand interactions can therefore be used in initial screening for interacting ligands from large chemical libraries, as well as during optimization of an initial ligand into a candidate drug. Further, it is important to understand the interaction of a drug with other proteins (so called “off target interactions”) where such interactions may result in side effects of treatments.

Methods disclosed herein comprise methods of assaying a chemical compound for the ability of the chemical agent to interact with or to influence a receptor and its coupled proteins, for example, an alpha 7nACh receptor and one or more coupled G proteins. Thus, an aspect of the disclosure relates to a method of assaying a chemical compound for ability to influence a receptor or receptor subunit, comprising the steps of: (a) introducing (i) a nucleic acid encoding a receptor or receptor subunit and/or proteins capable of coupling with the receptor or receptor subunit; and (b) exposing an expressed receptor or receptor subunit to a chemical compound; and, (c) evaluating the expressed and exposed receptor subunit to determine if the chemical compound influences the receptor subunit.

In an aspect, the influence of the chemical compound on the receptor or receptor subunit can be evaluated by measuring binding affinity or downstream signaling of the compound to the receptor or receptor subunit. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, Southwestern analysis, ELISA, and the like, which are described in, for example, Current Protocols in Molecular Biology (1999, John Wiley & Sons, NY), which is incorporated herein by reference in its entirety. Downstream signaling can be determined cell signaling assays which are well known to the skilled artisan, including, but not limited to, cellular calcium measures, cAMP detection, kinase activation, actin motility, and the like.

The agents to be screened include any compounds and are not limited to, those that are extracellular, intracellular, or of biologic or chemical origin. The methods of the disclosure also embrace ligands that optionally may be attached to a label, such as a radiolabel, a fluorescence label, a chemiluminecent label, an enzymatic label or an immunogenic label. The nucleic acids employed in such a test may either be free in solution, attached to a solid support, borne on a cell surface or located intracellularly or associated with a portion of a cell. One skilled in the art can, for example, measure the formation of complexes between receptor and/or receptor subunits and the compound being tested.

Alternatively, one skilled in the art can examine the diminution in complex formation between receptor subunits and its substrate caused by the compound being tested. Additionally, the present assays are suited to the development of high-throughput screens where detection may be carried out using for example a CCD camera, a luminometer, or any other suitable light detection system. In this manner, cells may be provided for example in multi-well plates to which test substances and reagents necessary for the detection of intracellular calcium may be added. Moreover, commercially available instruments such as “FLIPR-fluorimetric imaging bases plate reader” (Molecular Devices Corp, Sunnyvale, Calif., USA; Wood et al., 2000) and “VIPR” voltage ion probe reader (Aurora, Bioscience Corp. CA, USA) may be used. Very precise measurement of cellular fluorescence in a high throughput whole cell assay has become possible with the “FLIPR has shown considerable utility in measuring membrane potential of mammalian cells using voltage-sensitive fluorescent dyes but is useful for measuring essentially any cellular fluorescence phenomenon. The device uses low angle laser scanning illumination and a mask to selectively excite fluorescence within approximately 200 microns of the bottoms of the wells in standard 96 well plates. The low angle of the laser reduces background by selectively directing the light to the cell monolayer. This avoids background fluorescence of the surrounding media. This system then uses a CCD camera to image the whole area of the plate bottom to measure the resulting fluorescence at the bottom of each well. The signal measured is averaged over the area of the well and thus measures the average response of a population of cells. The system has the advantage of measuring the fluorescence in each well simultaneously thus avoiding the imprecision of sequential measurement well by well measurement. The system is also designed to read the fluorescent signal from each well of a 96 or 384 well plate as fast as twice a second. This feature provides FLIPR with the capability of making very fast measurements in parallel. This property allows for the measurement of changes in many physiological properties of cells that can be used as surrogated markers to a set of functional assays for drug discovery. FLIPR is also designed to have state of the art sensitivity. This allows it to measure very small changes with great precision.

Generally a plurality of assay mixtures are run in parallel with different agent concentrations to obtain a differential response to the various concentrations of candidate agent. Typically, one of these concentrations serves as a negative control, i.e. no compound. In an aspect, a high throughput screening protocol is employed, in which a large number of candidate compounds are tested in parallel using a large number of organisms. By “large number” is meant a plurality, where plurality means at least 10 to 50, usually at least 100, and more usually at least 1000, where the number of may be 10,000 or 50,000 or more, but in many instances will not exceed 5000.

Disclosed methods find use in the screening of a variety of different potentially candidate compounds. Candidate compounds can include molecules, peptides, proteins, nucleic acids, glycoproteins, lipids, lipoproteins, and other ligands that can bind to a receptor or receptor subunit. Candidate compounds may include numerous chemical classes, though typically they are organic molecules, for example, small organic compounds having a molecular weight of more than 50 and less than about 2,500 daltons. Candidate compounds comprise functional groups necessary for structural interaction with proteins, particularly hydrogen bonding, and may include an amine, carbonyl, hydroxyl or carboxyl group, or at least two of the functional chemical groups. The candidate compounds often comprise cyclical carbon or heterocyclic structures and/or aromatic or polyaromatic structures substituted with one or more of the above functional groups. Candidate compounds are also found among biomolecules including, but not limited to: peptides, saccharides, fatty acids, steroids, purines, pyrimidines, derivatives, structural analogs or combinations thereof.

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological compounds may be subjected to directed or random chemical modifications, such as acylation, alkylation, esterification, amidification, etc. to produce structural analogs. New potential pesticidal or therapeutic compounds may also be created using methods such as rational drug design or computer modeling.

The screening methods may be part of a multi-step screening process of evaluating candidate compounds for their efficacy in affecting receptor and or G protein molecules. In multi-step screening processes of the subject disclosure, a candidate compound or library of compounds is subjected to screening. In addition, a pre in vivo screening step may be employed, in which the compound is first subjected to an in vitro screening assay for its potential as an effective agonist or antagonist of disclosed receptors. Any convenient in vitro screening assay may be employed, where a variety of suitable in vitro screening assays are known to those of skill in the art.

Compositions and Methods Comprising Nucleic Acids

Compositions comprise nucleic acid constructs disclosed herein. In an aspect of the disclosure, the gene for a receptor, receptor subunit, or a G protein, is preceded by a reporter gene, such as a fluorescent protein gene (e.g., GFP, RFP, BFP, YFP, or dsRED2) or a luciferase protein gene, comprising a strong transcriptional stop-site, which is flanked by site specific recombinase recognition sites (e.g., Flox, Lox, or FRT-sites). A ubiquitous gene promoter (e.g., EF1-alpha or beta-actin) may drive expression of the “Loxed,” “Floxed” or “FRPed” reporter gene. A second gene product (e.g., a receptor subunit gene) is adjacent to the reporter gene but is not expressed in the absence of recombinase protein expression because of the strong transcription stop-site within reporter gene. However, when the recombinase protein expression is activated in the cells, the Loxed, Floxed, or FRPed reporter gene product is excised, and the second gene is juxtaposed to the ubiquitous gene promoter. Additionally, tissue-specific recombination may be facilitated by laser-activation of a heat-shock inducible site-specific recombinase transgene through use of a laser. Laser activation may be targeted to individual cells during embryologic development.

Various methods are known in the art for introducing nucleic acids into host cells. One method is microinjection, in which DNA is injected directly into the nucleus of cells through fine glass needles (or RNA is injected directly into the cytoplasm of cells). Alternatively, DNA can be incubated with an inert carbohydrate polymer (dextran) to which a positively charged chemical group (DEAE, for diethylaminoethyl) has been coupled. The DNA sticks to the DEAE-dextran via its negatively charged phosphate groups. These large DNA-containing particles stick in turn to the surfaces of cells, which are thought to take them in by a process known as endocytosis. Some of the DNA evades destruction in the cytoplasm of the cell and escapes to the nucleus, where it can be transcribed into RNA like any other gene in the cell. In another method, cells efficiently take in DNA in the form of a precipitate with calcium phosphate. In electroporation, cells are placed in a solution containing DNA and subjected to a brief electrical pulse that causes holes to open transiently in their membranes. DNA enters through the holes directly into the cytoplasm, bypassing the endocytotic vesicles through which they pass in the DEAE-dextran and calcium phosphate procedures (passage through these vesicles may sometimes destroy or damage DNA). DNA can also be incorporated into artificial lipid vesicles, liposomes, which fuse with the cell membrane, delivering their contents directly into the cytoplasm. In an even more direct approach, used primarily with plant cells and tissues, DNA is absorbed to the surface of tungsten microprojectiles and fired into cells with a device resembling a shotgun.

Several of these methods, microinjection, electroporation, and liposome fusion, have been adapted to introduce proteins into cells. For review, see Mannino and Gould-Fogerite, 1988; Shigekawa and Dower, 1988; Capecchi; 1980 and Klein et al., 1987.

Further methods for introducing nucleic acids into cells involve the use of viral vectors. Since viral growth depends on the ability to get the viral genome into cells, viruses have devised clever and efficient methods for doing it. One such virus widely used for protein production is an insect virus, baculovirus. Baculovirus attracted the attention of researchers because during infection, it produces a crystal protein which encloses multiple virions (polyhedrin protein) to spectacular levels. If a foreign gene were to be substituted for this viral gene, it too ought to be produced at high level. Baculovirus, like Vaccinia, is very large, and therefore foreign genes must be placed in the viral genome by recombination. To express a foreign gene in baculovirus, the gene of interest is cloned in place of the viral coat protein gene in a plasmid carrying a small portion of the viral genome. The recombinant plasmid is cotransfected into insect cells with wild-type baculovirus DNA. At a low frequency, the plasmid and viral DNAs recombine through homologous sequences, resulting in the insertion of the foreign gene into the viral genome. Virus plaques develop, and the plaques containing recombinant virus look different because they lack polyhedrin crystals. The plaques with recombinant virus are picked and expanded. This virus stock is then used to infect a fresh culture of insect cells, resulting in high expression of the foreign protein. For a review of baculovirus vectors, see Miller (1989). Various viral vectors have also been used to transform mammalian cells, such as bacteriophage, vaccinia virus, adenovirus, and retrovirus.

As indicated, some of these methods of transforming a cell require the use of an intermediate plasmid vector. U.S. Pat. No. 4,237,224 to Cohen and Boyer describes the production of expression systems in the form of recombinant plasmids using restriction enzyme cleavage and ligation with DNA ligase. These recombinant plasmids are then introduced by means of transformation and replicated in unicellular cultures including procaryotic organisms and eucaryotic cells grown in tissue culture. The DNA sequences are cloned into the plasmid vector using standard cloning procedures known in the art, as described by Sambrook and Russell (2000).

In aspects of the present disclosure, host cells are utilized which endogenously produce an accessory protein and, accordingly, it is unnecessary to separately introduce nucleic acid encoding the accessory protein into the host cell. Thus, these embodiments relate to a method of assaying a chemical compound for ability to influence a receptor subunit, comprising the steps of: (a) introducing (i) the nucleic acid sequence encoding the receptor subunit into a host cell in vitro to express the receptor subunit, wherein an accessory protein is endogenously produced and expressed by the host cell, and wherein the host cell is capable of responding to a spinosyn; and thereafter, (b) exposing the expressed receptor subunit to a chemical compound; and, (c) evaluating the exposed receptor subunit to determine if the chemical compound influences the receptor subunit.

In any event, the host cells according to the present disclosure can be exposed to various chemical compounds and evaluated for their interaction with these compounds to develop and identify new receptor affecting compounds. In embodiments of the present disclosure, the chemical compound is a mixture of chemical compounds. The evaluation of the exposed host cell to determine if the chemical compound influences the receptor subunit can be by any means known in the art.

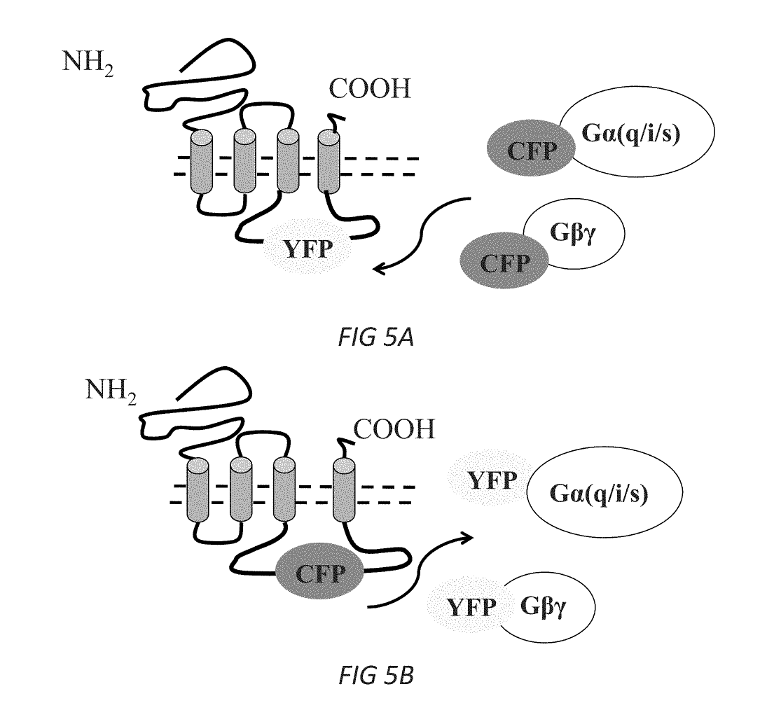

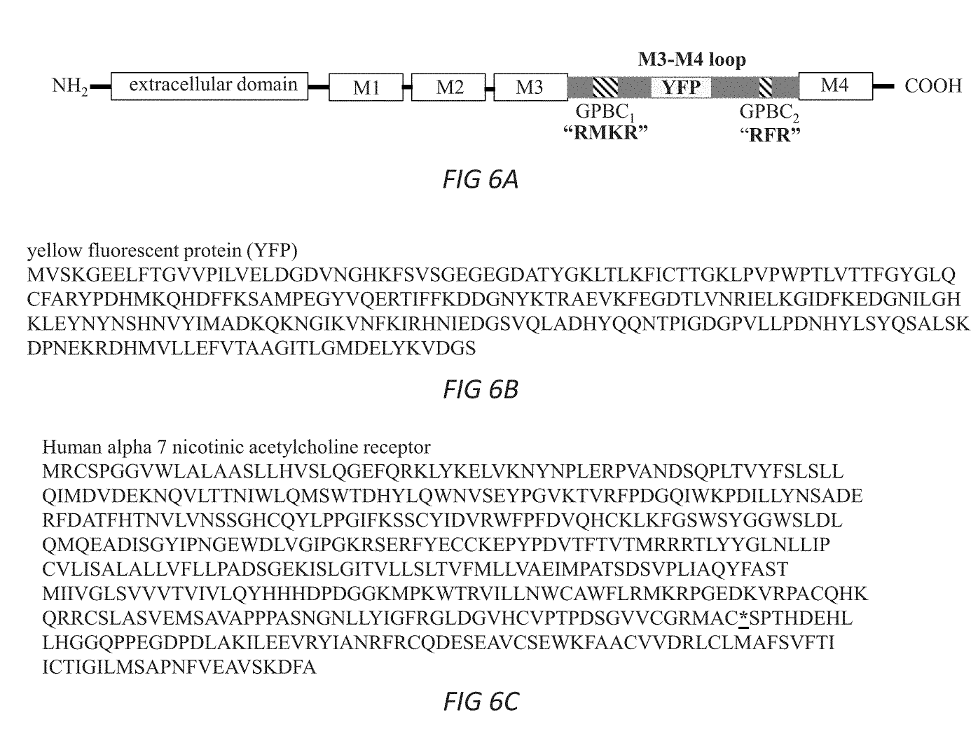

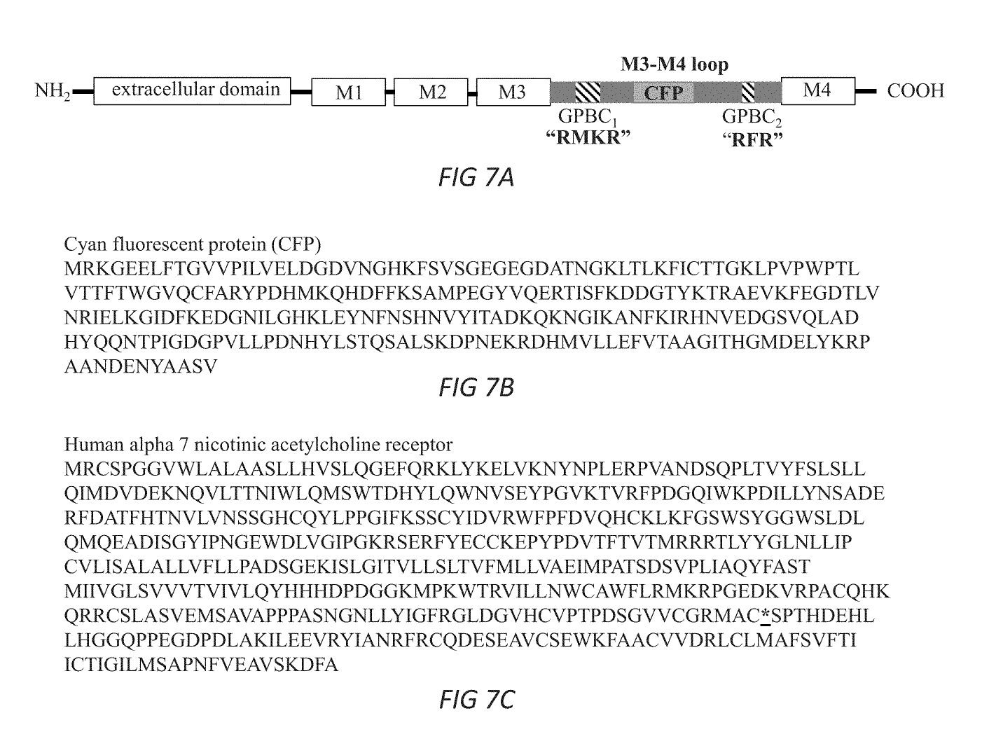

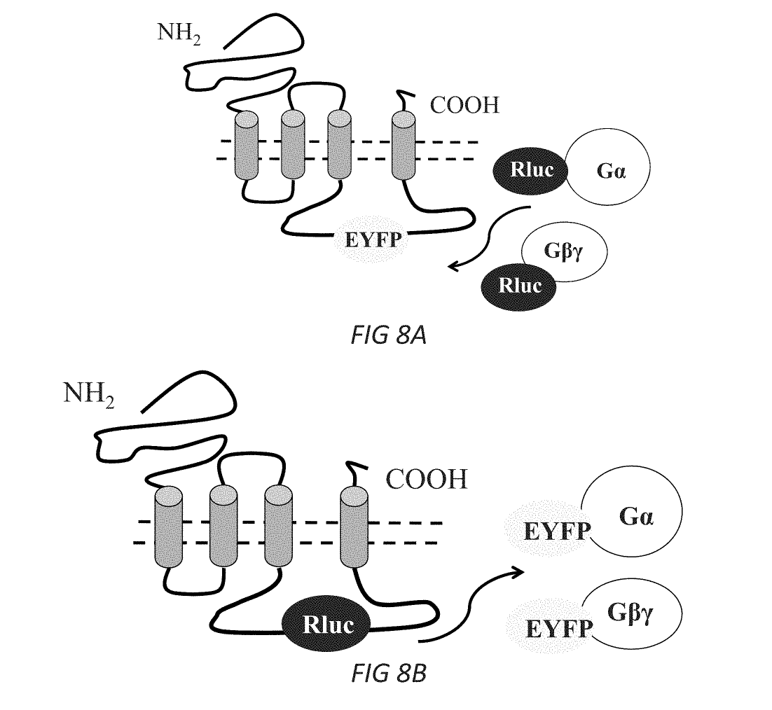

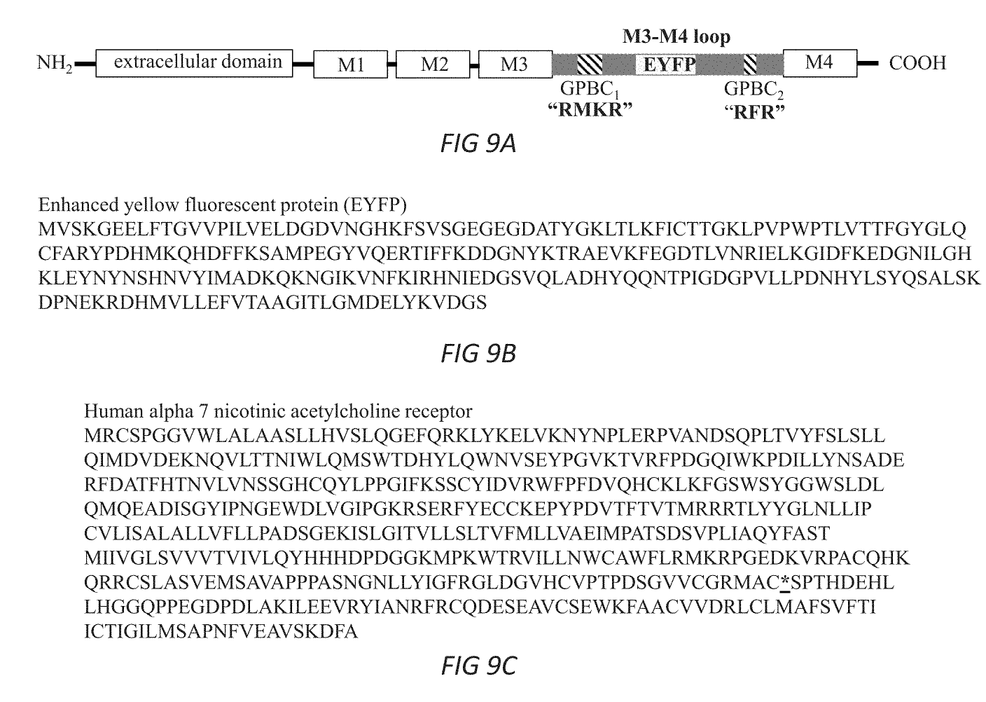

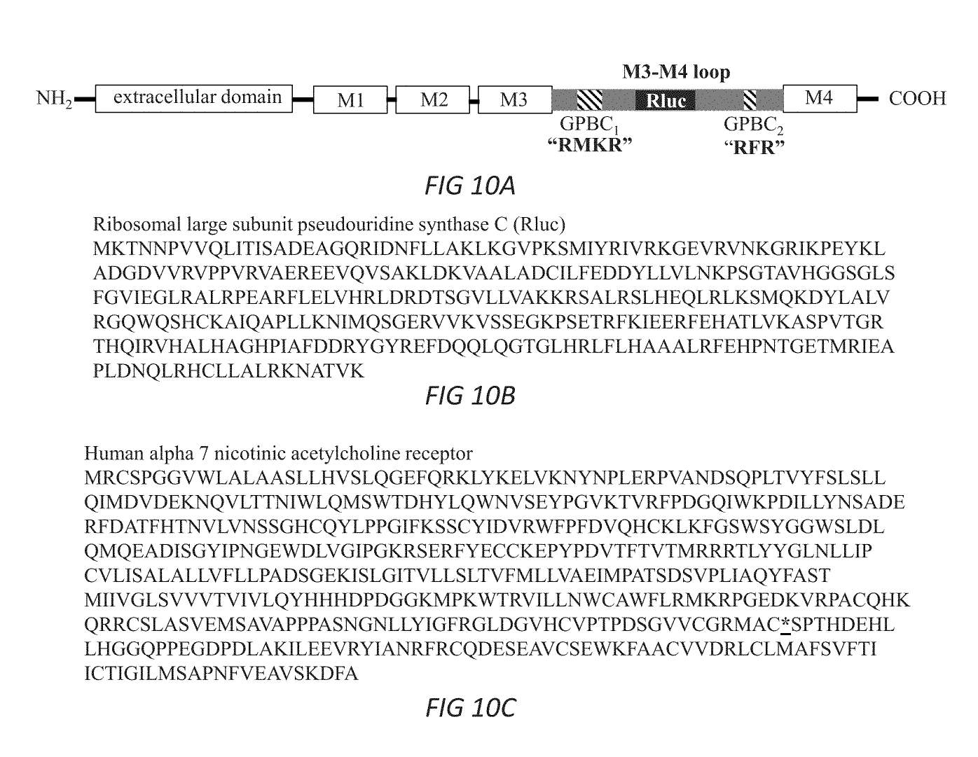

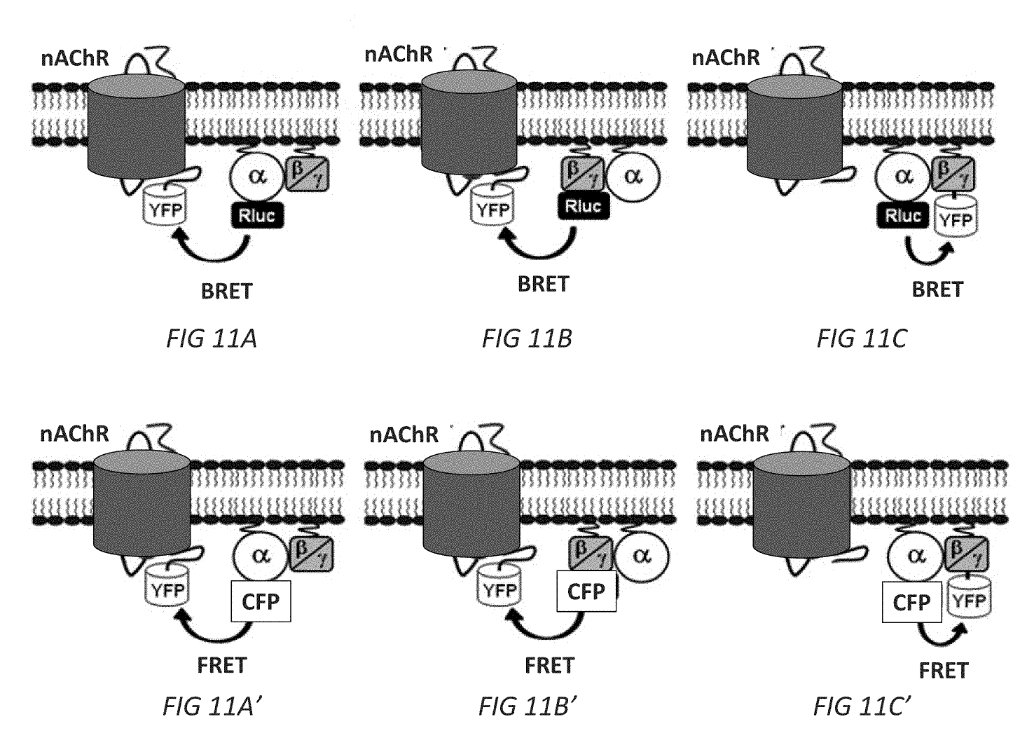

Detection and Labeling of Receptors, Coupled Proteins or Both (See, e.g., FIGS. 5-11)

FRET (Fluorescence Resonance Energy Transfer) is based on the transfer of energy between two fluorophores, a donor and an acceptor, when in close proximity Molecular interactions between biomolecules can be assessed by coupling each partner with a fluorescent label and by detecting the level of energy transfer. When two entities come close enough to each other, excitation of the donor by an energy source (e.g. a flash lamp or a laser) triggers an energy transfer towards the acceptor, which in turn emits specific fluorescence at a given wavelength. The donor and acceptor can be grafted covalently onto multiple partners that can associate, among others, two dimerizing proteins, two DNA strands, an antigen and an antibody, or a ligand and its receptor.

Because of these spectral properties, FRET, a donor-acceptor complex, can be detected without the need for physical separation from the unbound partners. The term “FRET” means “fluorescence resonance energy transfer” or “Forster resonance energy transfer”, and refers to the radiationless transmission of an energy quantum from its site of absorption (the donor) to the site of its utilization (the acceptor) in a molecule, or system of molecules, by resonance interaction between donor and acceptor species, over distances considerably greater than interatomic, without substantial conversion to thermal energy, and without the donor and acceptor coming into kinetic collision. A donor is a moiety that initially absorbs energy (e.g., optical energy or electronic energy). A luminescent metal complex as described herein can comprise two donors: 1) an organic antenna moiety, which absorbs optical energy (e.g., from a photon); and 2) a lanthanide metal ion, which absorbs electronic energy (e.g., transferred from an organic antenna moiety).

For FRET to occur successfully, several conditions are met: Proximity The donor and acceptor fluorophores must be close to one another for the FRET process to be efficient. FRET efficiency (E) is defined by the equation E=Ro6/(Ro6+r6), where Ro is the Förster radius and r is the actual distance between the two fluorophores. The Förster radius is the distance at which 50% of the excitation energy is transferred from the donor to the acceptor, and the Ro value usually lies between 10-100 Å (1-10 nm). FRET pairs with an Ro value towards the higher end of this range are often preferred due to the increased likelihood of FRET occurrence.

Spectral overlap. The emission spectrum of the donor fluorophore must overlap the absorption spectrum of the acceptor fluorophore. The greater the degree of spectral overlap, the more likely FRET is to occur.

Bioluminescence resonance energy transfer (BRET) has become a widely used technique to monitor protein-protein interactions. It involves resonance energy transfer between a bioluminescent donor and a fluorescent acceptor. Because the donor emirs photons intrinsically, fluorescence excitation is unnecessary. Therefore, BRET avoids some of the problems inherent in fluorescence resonance energy transfer (FRET) approaches, such as photobleaching, autofluorescence, and undesirable stimulation of photo-biological processes. In the past, BRET signals have generally been too dim to be effectively imaged. Newly available cameras that are more sensitive coupled to image splitter now enable BRET imaging in plant and mammalian cells and tissues In addition, new substrates and enhanced luciferases enable brighter signals that allow even subcellular BRET imaging.

Generally, the nomenclature used herein and many of the fluorescence, luminescence, computer, detection, chemistry, and laboratory procedures described herein are commonly employed in the art. Standard techniques are generally used for chemical synthesis, fluorescence or luminescence monitoring and detection, optics, molecular biology, and computer software and integration. Chemical reactions, cell assays, and enzymatic reactions are typically performed according to the manufacturer's specifications where appropriate. See, generally, Lakowicz, J. R. Topics in Fluorescence Spectroscopy, (3 volumes) New York: Plenum Press (1991), and Lakowicz, J. R. Emerging applications of florescence spectroscopy to cellular imaging: lifetime imaging, metal-ligand probes, multi photon excitation and light quenching, Scanning Microsc. Suppl. Vol. 10 (1996) pages 213-24, for fluorescence techniques; Sambrook et al., Molecular Cloning: A Laboratory Manual, 2ed. (1989) Cold Spring Harbor Laboratory Press, Cold Spring Harbor, N.Y., for molecular biology methods; Cells: A Laboratory Manual, 1.sup.st edition (1998) Cold Spring Harbor Laboratory Press, Cold Spring Harbor, N.Y., for cell biology methods; and Optics Guide 5 Melles Griot® Irvine Calif., and Optical Waveguide Theory, Snyder & Love (published by Chapman & Hall) for general optical methods, all of which are incorporated herein by reference.

General methods for performing a variety of fluorescent or luminescent assays on luminescent materials are known in the art and are described in, e.g., Lakowicz, J. R., Topics in Fluorescence Spectroscopy, volumes 1 to 3, New York: Plenum Press (1991); Herman, B., Resonance Energy Transfer Microscopy, in Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361; and Bernard Valeur, “Molecular Fluorescence: Principles and Applications” Wiley VCH, 2002. Guidance in the selection and use of specific resonance acceptor moieties is available at, for example, Berlman, I. B., Energy transfer parameters of aromatic compounds, Academic Press, New York and London (1973), which contains tables of spectral overlap integrals for the selection of resonance energy transfer pairs. Additional information sources include the Molecular Probes Catalog (2003) and website; and Tsien et al., 1990 Handbook of Biological Confocal Microscopy, pp. 169-178. Instruments useful for performing FP and/or RET and TR-RET applications are available from Tecan Group Ltd. (Switzerland) (Ultra, Ultra 384, Ultra Evolution); Perkin-Elmer (Boston, Mass.) (Fusion, EnVision, Victor V, and ViewLux), Amersham Bioscience (Piscataway, N.J.) (LeadSeeker); and Molecular Devices Corporation (Sunnyvale, Calif.) (Analyst AD, GT, and HT).

The term “acceptor” refers to a chemical or biological moiety that accepts energy via resonance energy transfer. In FRET applications, acceptors may re-emit energy transferred from a donor fluorescent or luminescent moiety as fluorescence and are “fluorescent acceptor moieties.” As used herein, such a donor fluorescent or luminescent moiety and an acceptor fluorescent moiety are referred to as “a pair.” Examples of acceptors include coumarins and related fluorophores; xanthenes such as fluoresceins and fluorescein derivatives; fluorescent proteins such as GFP and GFP derivatives; rhodols, rhodamines, and derivatives thereof; resorufins; cyanines; difluoroboradiazaindacenes; and phthalocyanines Acceptors, including fluorescent acceptor moieties, can also be useful as fluorescent probes in fluorescence polarization assays.

Examples of molecules used in FRET assays include, but are not limited to, single chain (single-stranded intrachain) FRET molecules incorporated into an aptamer population via their nucleotide triphosphate derivatives (for example, ALEXFLUOR-NTPs, CASCADE BLUE-NTPs, CHROMATIDE-NTPs, fluorescein-NTPs, rhodamine-NTPs, RHODAMINE GREEN-NTPs, tetramethylrhodamine-dNTPs, OREGON GREEN-NTPs, and TEXAS RED-NTPs may be used to provide the fluorophores, while dabcyl-NTPs, Black Hole Quencher or BHQ-NTPs, and QSY dye-NTPs may be used for the quenchers). This process is generally referred to as “doping” with F-NTPs and Q-NTPs. These dyes, not in the NTP derivative form, may be covalently linked to receptor proteins, receptor subunit proteins, and/or G proteins.

Exemplary BRET dyes include, but are not limited to, YFP and RLuc.

Disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits can include receptors and/or coupled proteins, cells comprising such receptors and/or proteins, cells expressing such receptors and/or proteins, nucleic acid constructs or vectors encoding such receptors and/or proteins, labeled receptors and/or proteins, and reagents for assays comprising such receptors and/or proteins.

The kits can include instructions for using the reagents described in the methods disclosed herein.

### Definitions

It must be noted that as used herein and in the appended claims, the singular forms “a”, “an”, and “the” include plural reference unless the context clearly dictates otherwise. Thus, for example, reference to “a therapeutic” includes a plurality of such therapeutics, reference to “the assay component” is a reference to assay components known to those skilled in the art, and so forth.

Patents and references referred to in this disclosure are herein specifically incorporated in their entireties.

The terms “label” or “labeled” refer to a detectable molecule that is generally attached to a nucleic acid or protein so that the presence or activity of the nucleic acid or protein can be detected and/or measured. For example, the inclusion of a luminescent metal complex or a fluorescent acceptor moiety on molecule or substance. A label includes pairs of detectable molecules.

The term “covalent” refers to a form of chemical bonding that is characterized by the sharing of pairs of electrons between atoms, or between atoms and other covalent bonds.

“Optional” or “optionally” means that the subsequently described event, circumstance, or material may or may not occur or be present, and that the description includes instances where the event, circumstance, or material occurs or is present and instances where it does not occur or is not present.

“Binding affinity” refers to the propensity of a ligand to interact with a receptor or other protein.

The phrase “cell surface receptor that binds to an intracellular binding partner” refers generally to a protein or a protein complex on the surface of a cell, such as a eukaryotic cell, that serves as a receptor in the sense of recognizing a specific ligand, and, further, that operates through binding to a second molecule that forms a complex with the receptor under certain conditions, such as receptor activation. Thus, the term includes cell surface proteins such as G coupled protein receptors (GPCR), for example, and G protein coupled channel receptors such as the alpha 7 nAChR.

The term “test agent” “test agent”, “test compound”, “agent”, or “compound” as used herein refers to any molecule or compound, which is tested in the methods of the disclosure to determine if it effects the receptor or receptor subunit, the one or more coupled proteins, the relationship between the receptor and the coupled proteins, or more than one of these. Alternatively viewed, the test agent is a potential ligand for the receptor or receptor subunit, and/or the one or more coupled proteins. Thus, the test agent or ligand may be a protein, polypeptide, peptide, small molecule, RNA, or DNA molecule. In a particular embodiment, the test agent may be a drug or pharmaceutical product, a cell metabolite or a hormone. The test agent or ligand may be naturally occurring or may be synthetically or recombinantly produced, using any of the methods known to those of skill in the art.

The test agent used may or may not bind to the receptor or receptor subunit and/or the one or more coupled proteins; in one aspect the method of the disclosure determines or assesses whether a particular molecule or compound is capable of binding to the receptor or receptor subunit and/or the one or more coupled proteins, e.g., whether a test agent or compound is a ligand. Thus, the disclosure can be used to screen a small molecule library for molecules which are capable of binding to the receptor or receptor subunit and/or the one or more coupled proteins, whether at the active binding site or elsewhere on the protein. Some of the molecules tested may not bind, whereas others may bind to the receptor or receptor subunit and/or the one or more coupled proteins. Additionally, a method of the disclosure can be used to identify variants of small molecules known to bind to the receptor or receptor subunit, which can bind the receptor or receptor subunit and/or the one or more coupled proteins with higher affinity (or alternatively with lower affinity). Thus, test agents can be mutated ligands or known (or unknown) the receptor or receptor subunit the receptor or receptor subunit and/or the one or more coupled proteins binding partners. The production of such mutated molecules is achieved by using any of the mutation processes known to those of skill in the art.

The term “ligand” as used herein refers to a test agent or more generally to a compound which is capable of binding to the receptor or receptor subunit and/or the one or more coupled proteins. The ligand of interest may bind elsewhere on the protein or may compete for binding e.g. with a physiological ligand. Ligands of interest may be drugs or drug candidates or naturally occurring binding partners, physiological substrates, etc. Thus, the ligand can bind to the receptor or receptor subunit and/or the one or more coupled proteins to form a larger complex. The ligand can bind to the receptor or receptor subunit and/or the one or more coupled proteins with any affinity i.e. with high or low affinity.

Hence, when a test agent is already known to bind the receptor or receptor subunit and/or the one or more coupled proteins (and thus is a ligand for the receptor or receptor subunit and/or the one or more coupled proteins), the method of the disclosure can be used to assess the binding of the ligand to the receptor or receptor subunit and/or the one or more coupled proteins e.g. to determine the strength of the interaction.

“Substantially similar” refers to nucleic acid fragments wherein changes in one or more nucleotide bases result in substitution of one or more amino acids, but do not affect the functional properties of the protein encoded by the DNA sequence. “Substantially similar” also refers to nucleic acid fragments wherein changes in one or more nucleotide bases do not affect the ability of the nucleic acid fragment to mediate alteration of gene expression by antisense or co-suppression technology. “Substantially similar” also refers to modifications of nucleic acid fragments such as deletion or insertion of one or more nucleotides that do not substantially affect the functional properties of the resulting transcript vis-a-vis the ability to mediate alteration of gene expression by antisense or co-suppression technology or alteration of the functional properties of the resulting protein molecule. It is therefore understood that the disclosure encompasses more than the specific exemplary sequences.

For example, it is well known in the art that antisense suppression and co-suppression of gene expression may be accomplished by using nucleic acid fragments representing less than the entire coding region of a gene, and by nucleic acid fragments that do not share 100% sequence identity with the gene to be suppressed. Moreover, alterations in a gene which result in the production of a chemically equivalent amino acid at a given site, but do not affect the functional properties of the encoded protein, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

Moreover, substantially similar nucleic acid fragments may also be characterized by their ability to hybridize, under stringent conditions (0.1.times.SSC, 0.1% SDS, 65.degree. C.), with the nucleic acid fragments disclosed herein.

Substantially similar nucleic acid fragments of the instant disclosure may also be characterized by the percent similarity of the amino acid sequences that they encode to the amino acid sequences disclosed herein, as determined by algorithms commonly employed by those skilled in this art. Preferred are those nucleic acid fragments whose nucleotide sequences encode amino acid sequences that are 80% similar to the amino acid sequences encoded by the nucleic acid sequences reported herein. More preferred nucleic acid fragments encode amino acid sequences that are 90% similar to the amino acid sequences encoded by the nucleic acid sequences reported herein. Most preferred are nucleic acid fragments that encode amino acid sequences that are 95% similar to the amino acid sequences encoded by the nucleic acid sequences reported herein. Sequence alignments and percent similarity calculations were performed using programs from the Vactor NTi Suite (InforMax, North Bethesda, Md.). Multiple alignments of the sequences were performed using the Clustal method of alignment (Higgins and Sharp, 1989) with the default parameters (GAP PENALTY=10, GAP extension PENALTY=0.1) (hereafter, Clustal algorithm). Default parameters for pairwise alignments using the Clustal method were KTUPLE 1, GAP PENALTY=3, WINDOW=5 and DIAGONALS SAVED=5.

A “substantial portion” of an amino acid or nucleotide sequence refers to enough of the amino acid sequence of a polypeptide or the nucleotide sequence of a gene to afford putative identification of that polypeptide or gene, either by manual evaluation of the sequence by one skilled in the art, or by computer automated sequence comparison and identification using algorithms such as BLAST (Basic Local Alignment Search Tool; Altschul et al., 1993;). In general, a sequence of ten or more contiguous amino acids or thirty or more nucleotides is necessary to putatively identify a polypeptide or nucleic acid sequence as homologous to a known protein or gene. Moreover, with respect to nucleotide sequences, gene specific oligonucleotide probes comprising 20 30 contiguous nucleotides may be used in sequence dependent methods of gene identification (e.g., Southern hybridization) and isolation (e.g., in situ hybridization of bacterial colonies or bacteriophage plaques). In addition, short oligonucleotides of 12 15 bases may be used as amplification primers in PCR in order to obtain a particular nucleic acid fragment comprising the primers. Accordingly, a “substantial portion” of a nucleotide sequence comprises enough of the sequence to afford specific identification and/or isolation of a nucleic acid fragment comprising the sequence. The instant specification teaches partial or complete amino acid and nucleotide sequences encoding one or more particular plant proteins. The skilled artisan, having the benefit of the sequences as reported herein, may now use all or a substantial portion of the disclosed sequences for purposes known to those skilled in this art.

“Transcription regulatory region” and “regulatory region” refer to the section of DNA which regulates gene transcription. A regulatory region may include a variety of cis-acting elements, including, but not limited to, promoters, enhancers and hormone response elements. Also, since introns and 5′ UTR have been known to influence transcription, a transcription regulatory region can include such sequences. A regulatory region may be operatively linked to a nucleic acid to ensure expression of the nucleic acid in a host cell.

“Transgenic animal” refers to an animal that has been modified by the artificial insertion, and stable integration, of DNA into its genome. The DNA may be inserted randomly or targeted to a specific site in a chromosome or an episomal or extrachromosomal element.

“Transgenic cell” refers to a cell containing artificially inserted DNA within a chromosome or an episomal or extrachromosomal element.

“Variant” refers to substantially similar sequences. Generally, nucleic acid sequence variants of the disclosure will have at least 46%, 48%, 50%, 52%, 53%, 55%, 60%, 65%, 70%, 75%, 76%, 77%, 78%, 79%, 80%, 85%, 86%, 87%, 88%, 89%, 90%, 91%, 92%, 93%, 94%, 95%, 96%, 97%, 98% or 99% sequence identity to the native nucleotide sequence, wherein the % sequence identity is based on the entire sequence and is determined by GAP 10 analysis using default parameters. Generally, polypeptide sequence variants of the disclosure will have at least about 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the native protein, wherein the % sequence identity is based on the entire sequence and is determined by GAP 10 analysis using default parameters. GAP uses the algorithm of Needleman and Wunsch (J. Mol. Biol. 48:443-453, 1970) to find the alignment of two complete sequences that maximizes the number of matches and minimizes the number of gaps.

“Variant” also refers to substantially similar sequences that contain amino acid sequences highly similar to the motifs contained within the disclosure and optionally required for the biological function of the disclosure. Generally, polypeptide sequence variants of the disclosure will have at least 85%, 90% or 95% sequence identity to the conserved amino acid residues in the defined motifs.

Standard recombinant DNA and molecular cloning techniques used herein are well known in the art and are described more fully in Sambrook and Russell (2000).

Variants included in the disclosure may contain individual substitutions, deletions or additions to the nucleic acid or polypeptide sequences which alter, add or delete a single amino acid or a small percentage of amino acids in the encoded sequence. A “conservatively modified variant” is an alteration which results in the substitution of an amino acid with a chemically similar amino acid. When the nucleic acid is prepared or altered synthetically, advantage can be taken of known codon preferences of the intended host.

The nucleic acid fragments of the instant disclosure may be used to isolate cDNAs and genes encoding homologous proteins from the same or other species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other nicotinic acetylcholine receptor subunits, either as cDNAs or genomic DNAs, could be isolated directly by using all or a portion of the instant nucleic acid fragments as DNA hybridization probes to screen libraries from any desired organism employing methodology well known to those skilled in the art. Specific oligonucleotide probes based upon the instant nucleic acid sequences can be designed and synthesized by methods known in the art (Sambrook and Russell, 2000). Moreover, the entire sequences can be used directly to synthesize DNA probes by methods known to the skilled artisan such as random primer DNA labeling, nick translation, or end-labeling techniques, or RNA probes using available in vitro transcription systems.

In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding genes. Alternatively, the second primer sequence may be based upon sequences derived from the cloning vector. For example, the skilled artisan can follow the RACE protocol (Frohman et al., 1988) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (Invitrogen, Madison, Wis.), specific 3′ or 5′ cDNA fragments can be isolated (Ohara et al., 1989; Loh et al., 1989). Products generated by the 3′ and 5′ RACE procedures can be combined to generate full-length cDNAs (Frohman and Martin, 1989). Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, 1984; Sambrook and Russell, 2000).

The present disclosure includes a plurality of polynucleotides that encode for the identical amino acid sequence. The degeneracy of the genetic code allows for such “silent variations” which can be used, for example, to selectively hybridize and detect allelic variants of polynucleotides of the present disclosure. Additionally, the present disclosure includes isolated nucleic acids comprising allelic variants. The term “allele” as used herein refers to a related nucleic acid of the same gene. A variant may also be described as, for example, a “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base.

Variants of nucleic acids included in the disclosure can be obtained, for example, by oligonucleotide-directed mutagenesis, linker-scanning mutagenesis, mutagenesis using the polymerase chain reaction, and the like. Also, see generally, McPherson (1991). Thus, the present disclosure also encompasses DNA molecules comprising nucleotide sequences that have substantial sequence similarity with the inventive sequences.

With respect to particular nucleic acid sequences, “conservatively modified variants” refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present disclosure is implicit in each described polypeptide sequence and is within the scope of the claimed disclosure.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of, from 1 to 50 can be so altered. Thus, for example, 1, 2, 3, 14, 25, 37, 45 or 50 alterations can be made. Conservatively modified variants typically provide similar biological activity as the unmodified polypeptide sequence from which they are derived. For example, substrate specificity, enzyme activity, or ligand/receptor binding is generally at least 20%, 30%, 40%, 50%, 60%, 70%, 80%, or 90% of the native protein for its native substrate. Conservative substitution tables providing functionally similar amino acids are well known in the art.

For example, the following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). Other acceptable conservative substitution patterns known in the art may also be used, (see Creighton, 1984); such as the scoring matrices of sequence comparison programs like the GCG package, BLAST, or CLUSTAL for example.

“Vector” refers to a nucleic acid molecule capable of transporting another nucleic acid to which it has been linked One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Ranges may be expressed herein as from “about” one particular value, and/or to “about” another particular value. When such a range is expressed, also specifically contemplated and considered disclosed is the range¬from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present disclosure is not entitled to antedate such disclosure by virtue of prior disclosure. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step. The broader term “comprising” also includes the more narrow terms of “consisting essentially of” and consisting of”.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a compound is disclosed and discussed and a number of modifications that can be made to the compound or drug are discussed, each and every combination and permutation of the compound and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The term receptor as used herein is intended to encompass subtypes of the named receptors, and mutants, such as constitutively active mutants, homologs thereof, and chimeric receptors including the nucleic acid encoding such receptors. Chimeric receptors as used herein refers to receptors which may be formed comprising parts of mammalian receptors found from different sources.

### EXAMPLES

### Example 1

Fluorescence Resonance Energy Transfer (FRET) is a well-established method useful in cellular microscopy. When two fluorophores (the “donor” and the “acceptor”) with overlapping emission/absorption spectra are within 80-50 Å of one another and their transition dipoles are appropriately oriented, the donor fluorophore is able to transfer its excited-state energy to the acceptor fluorophore. A series of alpha 7 nicotinic acetylcholine receptors (nAChR) and heterotrimeric G proteins (G proteins) linked to complementary fluorophores are used to test for changes in the interaction between the receptor and G protein complex under various experimental conditions including drug testing.

Bioluminescence resonance energy transfer (BRET) is also used for assaying protein-protein interactions with the same advantages of the FRET assay while avoiding the problems associated with fluorescence excitation (e.g. cell toxicity). In BRET, the donor fluorophore of the FRET pair is replaced by a luciferase (Rluc), in which bioluminescence from the luciferase in the presence of a substrate excites the acceptor fluorophore through the same resonance energy transfer mechanisms as FRET.

The two approaches are complementary and when used together can provide a powerful platform for testing changes in the interaction between the nAChR and the G protein in various cell lines under various treatment conditions. The construction of nAChR and G protein BRET/FRET pairs enables a new method for the screening of compounds and small molecules for their actions on the nAChR/G protein pathway in various types of cells. The BRET and FRET assays are conducted within various cell types including neural and immune cells. A mutant form of the alpha 7 nAChR that does not bind G proteins (King et al., 2015) is used as a negative control in the FRET/BRET assay.

Part 1. Cell Lines

1a. Fibroblast HEK-293T cells are grown in DMEM (Gibco) supplemented with 2 mM L-glutamine, 100 μg/ml sodium pyruvate, 100 units/ml penicillin/streptomycin, minimum Eagle's medium non-essential amino acid solution (1/100) and 5% (v/v) heat-inactivated FBS (all supplements were from Invitrogen).

1b. Neuroendocrine Pheochromocytoma line 12 (PC¬12) (ATCC® CRL¬1721™)) cells are grown on a poly-D-lysine (100 μg/ml) matrix and maintained in RPMI media (ATCC) supplemented with 10% horse serum, 5% fetal bovine serum, and 1% Penicillin Streptomycin (Thermo Fisher, Waltham, Mass., USA) (Nordman and Kabbani 2012). PC12 cells were differentiated by the addition of 2.5s mouse nerve growth factor (NGF) (Millipore, Billerica, Mass., USA) (50 ng/ml).

1c. Microglia EOC20 cells (ATCC® CRL-2469, Manassas, Va., USA) are grown on plastic petri dishes or glass coverslips (Genesee Scientific, San Diego, Calif., USA) coated with a poly-D-lysine (100 μg/ml) matrix and maintained in DMEM media (Thermo Fisher, Waltham, Mass., USA) supplemented with 10% fetal bovine serum and 1% Penicillin Streptomycin (Thermo Fisher). Mouse macrophage colony stimulating factor 1 (M-CSF1) added to the culture media (Pro Spec Bio, East Brunswick N.J., USA).

1d. Creation of stably transfected cell lines: Transfections to create the stably transfected cell line are done using 1 μg of cDNA (for the nAChR or the G protein) using the Effectene transfection reagent (Qiagen, Valencia, Calif.). Reagent components and DNA are mixed according to the manufacturer's instructions. Forty-eight hours post transfection, cells are trypsinized, resuspended in 2 mL of cell culture medium and seeded at low densities, from 1:100 to 1:1500 (v/v), in C-DMEM containing hygromycin B (130 mg/mL; Calbiochem, San Diego, Calif.). After 1-2 weeks, colonies are picked using cloning disks soaked in trypsin (0.5%)-EDTA (0.2%) solution then transferred to individual chambers in 24-well plates. Epifluorescence microscopy is employed 1-2 weeks later to screen expressing colonies for fluorescence. Clones are maintained for 6 or more passages and those that demonstrate a consistent level of fluorescence are selected for further propagation and cryopreservation.

Part 2. Vector Design

2a. BRET: Sequences encoding amino acid residues 1-155 and 155-238 of the Venus variant of YFP and amino acid residues 1-229 and 230-311 of Rluc8 protein subcloned into pcDNA3.1 vector to obtain YFP and Rluc hemitruncated proteins. Human cDNA for the alpha 7 nicotinic acetylcholine receptor (nAChR) is cloned into pcDNA3.1, amplified using sense and antisense primers harboring EcoRI and KpnI sites in the pRLuc vector (pRLuc-N1, PerkinElmer Life Sciences) or in the pEYFP vector (enhanced yellow variant of GFP, Clontech). Amplified fragments subcloned in-frame with restriction sites of pRLuc or pEYFP vectors at the alpha 7 nAChR M3-M4 loop (amino acid position of the human alpha 7 nAChR) provide plasmids for the nAChR protein fused to RLuc or YFP for the BRET assay. Primers for polymerase chain reaction (PCR) (see below) are designed to specifically amplify cDNA corresponding to the human α7 gene, including parts of 5′- and 3′-, non-coding regions, and to insert flanking restriction enzyme sites with 4-base leader sequences to facilitate hybridization at the restriction sites. The forward and reverse strand primer sequences respectively are:

5′-A TAT GGA TCC GGG ACA CGG CGG CTG CTC-3′
5′-G CGC TCT AGA CTA AGA TCT ACC CTG TAG G-3

Overlapping PCR is used with primers designed to insert the YFP or RLuc tag, in frame, into the M3-M4 intracellular loop of the human alpha 7 nAChR subunit. Three oligonucleotide segments are generated by separate PCR reactions, as follows: “S1,” encoding the human nAChR alpha 7 subunit from part of the 5′-flanking region through amino acid 412; “S2,” encoding the YFP or RLuc insert; and “S3,” encoding the α7 subunit from amino acid 413 through part of the 3′-flanking region. S1 and S3 are created using the above nAChR plasmid as a template. For S1, the forward primer is 5′-TAA TAC GAC TCA CTA TAG GG-3′ which hybridized to the T7 promoter region of the nAChR vector; the reverse primer, 5′-TGG GAC GTC ATA AGG ATA GCA GGC CAA ACG ACC ACA-3′ added an 18-base overlapping sequence for S2 to nucleotides corresponding to the 3′ region of S1. For S2, the forward primer, 5′-TGT GGT CGT TTG GCC TGC TAT CCT TAT GAC GTC CCA-3′ adds an 18-base sequence overlapping S1. The reverse primer, 5′-CTC ATC ATG TGT TGG GGA CTT GTA CAG CTC GTC CAT-3′ adds a 3′ sequence of 18 bases overlapping S3. The S3 forward primer, 5′-ATG GAC GAG CTG TAC AAG TCC CCA ACA CAT GAT GAG-3′ contains an 18-base overlapping sequence for S2; the reverse primer sequence is 5′-GAT TTA GGT GAC ACT ATA G-3′ which hybridizes to the nAChR vector. PCR settings: 35 cycles of PCR, 1 min at 95° C., 1 min 30 s at 55° C. and 1 min 30 s at 72° C., followed by a 4 min extension at 72° C. Segments are gel purified then overlapping PCR is used to join S2 and S3 at 1:10 molar ratio. PCR settings: 5 cycles of 1 min at 95° C., 1 min 30 s at 55° C. and 2 min at 72° C., are run with of 100 μL of SuperMix High Fidelity, 20 ng of S2 and 200 ng of S3. At the 5th cycle, the forward primer for S2 and the reverse primer for S3 were added (3 μL each, 10 μmol/L) and 30 additional cycles are run followed by a 4 min extension at 72° C. S1 is joined to (S2-S3) using the previous procedure, with an S1:(S2-S3) mass ratio of 1:10.

2b. FRET: FRET vectors are generated in the same manner as the BRET vectors described above. In FRET experiments however YFP and CFP fluorophore combinations are used rather than YFP and Rluc. In this case, YFP and CFP conjugated nAChR and G protein pairs are designed using essentially the same PCR based vector design method as for BRET (described above). The optimization of experimental conditions for PCR, ligation, transfection, etc. is done on a construct-by-construct basis.

Part 3. BRET Assay

3a. Cells are transiently co-transfected with a constant amount of cDNA encoding for the nAChR or the specific G protein fused to Rluc and with increasing amounts of cDNA corresponding to the receptor or the specific G protein fused to YFP. To control for cell number, the sample protein concentration is determined using a Bradford assay kit (Bio-Rad) using bovine serum albumin (BSA) dilutions as the standard. To quantify fluorescence, cells (20 μg protein) are distributed in 96-well microplates (black plates with a transparent bottom), and the fluorescence is read via a plate reader such as a Fluostar Optima fluorimeter (BMG Labtech, Offenburg, Germany) equipped with a high-energy xenon flash lamp using a 10-nm bandwidth excitation filter at 485 nm for detecting a BRET signal of the nAChR or G protein YFP reading.

3b. Fluorescence expression is quantified and determined as fluorescence of the sample minus the fluorescence of cells expressing the receptor or the G protein fused to Rluc alone. For BRET measurement, the equivalent of 20 μg of cell suspension is distributed in 96-well white microplates with white bottoms (Corning 3600, Corning, N.Y.) and 5 μM of coelenterazine H (for the YFP acceptor) (Molecular Probes, Eugene, Oreg.) added. Using coelenterazine H as substrates results in 485-nm emissions from Rluc, which allows the respective selective energy transfer to YFP. One minute after adding coelenterazine H, BRET is determined using the plate reader, which allows for the integration of the signal detected in the short-wavelength filter at 485 nm and the long-wavelength filter at 530 nm when YFP is the acceptor. To quantify receptor or G protein fused to Rluc expression, luminescence readings are typically performed after 10 min of adding 5 μM of coelenterazine H. A mutant form of the alpha 7 nAChR that does not bind G proteins (King et al., 2015) can also be used as a negative control in the BRET assay.

3c. Interaction within the BRET assay is measured as a saturation experiment in cells co-transfected with Rluc conjugated cDNA (1.5 μg) and increasing amounts of the YFP conjugated cDNA (0.5-3 μg). As negative controls, linear and low BRET values are obtained by transfecting cDNA corresponding to the “empty” Rluc vector (0.5 μg) as the BRET donor.

The relative amount of BRET in a given assay is used as a measure of the interaction between the G protein and the nAChR under the specific condition (e.g. ligand or small molecule application). This is typically expressed in assay result as a function of 100× the ratio between the fluorescence of the acceptor and the luciferase activity of the donor. Net BRET is defined as [(long-wavelength emission)/(short-wavelength emission)]−Cf, where Cf corresponds to [(long-wavelength emission)/(short-wavelength emission)] for the Rluc construct expressed alone in the same experiment. BRET is commonly expressed as milliBRET units and given as the means±S.D. of at least 4-5 separate experiments grouped as a function of the amount of BRET acceptor.

Part 4. FRET Assay

4a. Cell preparations in the FRET assay are similar to those in the BRET with a few minor modifications in the transfection. Specifically, cells are passaged 24 hr before transfection and 450,000 cells are transferred into one well of a six-well tissue culture plate for each fusion protein transfection. Cells are transfected with complementary FRET pair cDNA (YFP or CFP tagged nAChR or G protein) using Lipofectamine 2000 at (1.5 μg of each cDNA plasmid).

4b. FRET has been used previously to measure the kinetics and stoichiometry protein-protein binding for various protein types including both receptor and G proteins. Here FRET is adapted for the purpose of high throughput screening of compounds such as ligands and small molecules for their effect on nAChR-G protein interaction. In a typical assay, equimolar mixture (10 nM) of the two labeled proteins (the YFP or CFP labeled receptor or G protein) is excited at 485 nm to excite the donor molecule. Specific spectral changes relative to free protein are then measured under drug treatment or control conditions. Emission spectra between experimental groups (treated vs. non-treated, etc.) will be used to measure any significant spectral changes, which suggest energy transfer between the fluorophores and a shift in the interaction between the proteins. In this sense compounds can be tested for their ability to increase or decrease the interaction between the nAChR and the G protein in the FRET assay.

The following ratio is used to gauge binding between the receptor and the G protein in the FRET assay:

^{Iacceptor emission/Idonor emission}=IAF546/IAF488

This ratio corrects for instrumental fluctuations and helps minimize possible systematic errors in the fluorescence measurements.

4c. FRET is performed in either a plate reader or under direct live cell imaging using a confocal laser scanning microscope. Filters for FRET are blue diode (405 nm), Argon (458, 476, 488, 514 nm). green HeNe (543 nm), orange HeNe (594 nm), and red HeNe (633 nm) lasers. FRET analysis is ideal under environmentally controlled (temperature, humidity, and CO2) conditions. Experimental controls are critical to eliminate cross-talk or non-specific interaction between the YFP and CFP fluorophores. Negative FRET controls (using known non-interacting receptors and proteins) can also be useful to determine the background FRET that occurs due to over expression. A mutant form of the alpha 7 nAChR that does not bind G proteins (King et al., 2015) can also be used as a negative control in the FRET assay.

4c. FRET can result in both a decrease in fluorescence of the donor molecule as well as an increase in fluorescence of the acceptor, a ratio metric determination of the two signals can be made. The donor in most cases should be the protein of lower stoichiometry to minimize the percent of unpaired molecules. In the case of the nAChR and G protein FRET assay it is not yet clear which of the two proteins represents the optimal donor in the experiment.

Those skilled in the art will recognize, or be able to ascertain using no more than routine experimentation, many equivalents to the specific embodiments of the method and compositions described herein. Such equivalents are intended to be encompassed by the following claims.

What is claimed is:

1. An assay for detecting an effect a test agent has on a membrane receptor, comprising the steps of:
a) adding a test agent to a cell expressing a G-protein coupled membrane receptor/reporter protein coupled to one or more G proteins, wherein the receptor/reporter protein comprises a membrane receptor segment and a reporter segment comprising a first pair of reporter molecules, and wherein at least one of the one or more G proteins comprises a second pair of reporter molecules; and
b) detecting the signal from the first and/or second pair of reporter molecules, wherein a change in the signal indicates a change of coupling of the receptor/reporter protein and the at least one of the one or more G proteins.

2. The assay of claim 1, wherein the membrane receptor segment is the alpha7 nicotinic receptor and its variants.

3. The assay of claim 1, wherein the first pair of reporter molecules is YFP or CFP.

4. The assay of claim 3, wherein the second pair of reporter molecules is CFP or YFP.

5. The assay of claim 1, wherein the assay is further used to screen agents for their effect on membrane receptors.

6. The assay of claim 1, wherein the assay is further used to identify agents that disrupt normal membrane receptor interactions.

7. The assay of claim 1, wherein the test agent serves as an inverse agonist, antagonist, agonist, or allosteric modulator of the membrane receptor.

8. The assay of claim 7, wherein the inverse agonist, antagonist, agonist, or allosteric modulator of the membrane receptor is used in the study of receptor function.

9. The assay of claim 1, wherein the receptor/reporter protein is expressed from nucleic acid construct comprising a gene encoding the reporter segment that is fused in-frame to the 5′ or 3′ end of a gene encoding the membrane receptor segment.

10. The assay of claim 1, wherein the functionality of the membrane receptor segment is substantially unaffected by the presence of the reporter segment or a reporter molecule on the membrane receptor segment.

11. The assay of claim 1, wherein the signal is detected by FRET or BRET.

12. An assay for detecting a test agent which has an effect on a membrane receptor, comprising the steps of:
a) expressing a G-protein coupled membrane receptor/reporter protein capable of coupling to one or more G proteins in a cell, wherein the receptor/reporter protein comprises a membrane receptor segment and a reporter segment and wherein each of the one or more G proteins are labeled with a reporter molecule;
b) detecting a basal activity level of the reporter molecules;
c) adding a test agent to the cell; and
d) detecting a resulting activity level of the reporter molecules; and
e) comparing the basal activity level with the resulting activity level to determine whether alteration of the basal activity level has occurred, wherein the alteration is due to the test agent having an effect on the membrane receptor segment and/or the coupling with G proteins.

13. The assay of claim 12, wherein the membrane receptor segment is the alpha7 nicotinic receptor and its variants.

14. The assay of claim 13, wherein the reporter molecule is CFP or YFP.

15. The assay of claim 14, wherein the test agent serves as an inverse agonist, antagonist, agonist, or allosteric modulator of the membrane receptor.

16. The assay of claim 15, wherein the inverse agonist, antagonist, agonist, or allosteric modulator of the membrane receptor is used in the study of receptor function.

17. The assay of claim 12, wherein the receptor/reporter protein is expressed from nucleic acid construct comprising a gene encoding the reporter segment that is fused in-frame to the 5′ or 3′ end of a gene encoding the membrane receptor segment.

18. The assay of claim 12, wherein the functionality of the membrane receptor segment is substantially unaffected by the presence of the reporter segment or the reporter molecules.

19. The assay of claim 12, wherein the detecting of steps b) and d) is detected by BRET.

20. The assay of claim 12, wherein the detecting of steps b) and d) is detected by FRET.


SEQUENCE LISTING (2 sequences)

SEQ ID NO: 1 - DNA, 28 bp, artificial sequence
  Forward primer for alpha 7 nAChR M3-M4 loop
atatggatccgggacacggcggctgctc

SEQ ID NO: 2 - DNA, 29 bp, artificial sequence
  Reverse primer for alpha 7 nAChR M3-M4 loop
gcgctctagactaagatctaccctgtagg